(19) United States
(12) Patent Application Publication
(10) Pub. No.: US 2019/0175794 A1
(43) Pub. Date: Jun. 13, 2019

(54) MEDICAL DEVICES FOR CONTROLLABLE DRUG RELEASE

(71) Applicant: CAREFUSION 2200, INC., San Diego, CA (US)

(72) Inventors: Richard MENG, San Diego, CA (US); Theresa Hermel-Davidock, San Diego, CA (US); James Semler, San Diego, CA (US)

(21) Appl. No.: 15/837,925

(22) Filed: Dec. 11, 2017

(51) Int. Cl. A61L 29/08; A61L 29/10; A61L 29/16; A61L 29/14; A61K 47/69; A61K 47/56; A61K 33/38

(52) U.S. Cl. CPC A61L 29/085; A61L 29/106; A61L 29/16; A61L 29/146; A61L 29/145; A61K 47/6903; A61L 2300/104; A61K 33/38; A61L 2420/02; A61L 2420/06; A61L 2300/802; A61L 2300/62; A61L 2300/602; A61K 47/56

(57) ABSTRACT

Medical devices configured to provide a controllable release of a drug, particularly for use in pleural effusion therapy. The medical devices comprise a component including a carrier-drug complex, wherein the carrier-drug complex comprises one or more molecules of a drug reversibly bound to a porous carrier. The present disclosure also relates to methods of making the carrier-drug complexes and medical devices described herein.

### BACKGROUND

In certain medical treatments, it is helpful to provide a controlled release of one or more drugs into the body. For example, in pleural effusion therapy, it is helpful to use devices configured to provide a release of drug to the area being treated.

To provide such devices, current methods are difficult and lengthy. For example, current processes such as those described in U.S. Patent Publication Nos. 2012/025831 and 2016/0067385, may include applying a customized polymeric base coating layer to a device, drying and subsequently UV-curing the device, spraying a biologically active coat (comprising the drug) onto the device, drying, and then applying another polymeric base coating as a top coat. This process includes multiple steps with complicated machine controls and quality inspections in each step in order to meet product requirements, such as mechanical integrity, drug load, homogeneity, elution dynamic profile, etc. Moreover, when using silver ions (e.g., in the form of AgNO₃) as the drug, particularly when it is sprayed on the base coat through an aerosol or ultrasonic sprayer to form microscale AgNO₃ grains, it is critical, but also challenging, to control the AgNO₃ grain size and the grain size distribution and uniformity.

There is thus a need in the art for a more effective, more streamlined method for providing devices having a stable and controllable drug release pattern, particularly methods which are cost-effective, developmentally time efficient, and provide less complexity for production scaling-up. There is also a need in the art for devices having a controllable drug release rate and a longer product shelf life with an improved drug degradation resistance from environments, such as thermal, chemical, sterilization, and UV exposure. It would also be beneficial to provide a device wherein a drug, for example, silver ions may be consistently and uniformly provided on the surface thereof.

### SUMMARY

The present disclosure relates generally to medical devices configured to provide a controllable release of a drug. According to some aspects, the medical device comprises a component including a carrier-drug complex, wherein the carrier-drug complex comprises one or more molecules of a drug reversibly bound to a porous carrier. The present disclosure also relates to methods of making the carrier-drug complexes and medical devices described herein.

### BRIEF DESCRIPTION OF THE DRAWINGS

FIG. 1 shows an example of a carrier-drug complex according to the present disclosure.

FIG. 2 shows an example of a medical device and its preparation according to the present disclosure.

### DETAILED DESCRIPTION OF THE INVENTION

The present disclosure relates generally to medical devices configured to provide a controllable release of a drug. According to some aspects, the medical device comprises a component including a carrier-drug complex, wherein the carrier-drug complex comprises one or more molecules of a drug reversibly bound to a porous carrier. The present disclosure also relates to methods of making the carrier-drug complexes and medical devices described herein.

According to some aspects, the medical device may be configured for use in pleural effusion therapy. As used herein, the term “pleural effusion therapy” relates to any medical treatment that controls and/or eliminates excess fluid accumulation in the pleural cavity. Examples of such devices include, but are not limited to, catheters, chest tubes, syringes, needles, needleless connectors, injection ports, transfusion sets, antimicrobial catheter dressings, wound dressings and any devices having a surface suitable for coating with a carrier-drug complex. For example, the device may comprise a pleural catheter, such as the PLEURX® Pleural Catheter, marketed by Becton, Dickinson and Company. According to some aspects, the device may have one or more portions comprising any material suitable for medical device fabrication, such as a polymeric material. Examples of suitable materials include, but are not limited to, silicone, polyolefin, polystyrene, polyisoprene, polyisobutylene polyurethane, polyester, styrenic block copolymer, acrylic, acrylonitrile butadiene styrene (ABS), polycarbonate, polyvinyl chloride (PVC), hydrogel, and combinations thereof.

According to some aspects, the medical device may be provided with a component having a carrier-drug complex. For example, the medical device may comprise one or more coatings provided on a surface thereof, such as a surface of the device that interfaces with the pleural space during pleural effusion therapy. It should be understood that the component may be any component of the device such that the drug can interact with the pleural space.

The carrier-drug complex comprises one or more porous carriers. According to some aspects, the carrier may be configured to reversibly bind one or more drug molecules. As used herein, the term “reversibly bind” or “reversibly bound” refers to a non-permanent joining of two entities. It should be understood that reversible binding may comprise two entities whose joining exists in a state of equilibrium between molecules bound to one another and molecules not bound to one another.

According to some aspects, the carrier may reversibly bind the one or more drug molecules via an ionic interaction. For example, the carrier may comprise one or more ionic moieties capable of reversibly binding one or more drug molecules. Examples of ionic moieties according to the present disclosure include, but are not limited to, polystyrene sulfonic acid, carboxylic acid, polyacrylic acid, polyaspartic acid, amino-functionalized silica gel and combinations thereof.

According to some aspects, the carrier may reversibly bind the one or more drug molecules via loading into one or more pores of the carrier. As used herein, the term “pore” refers to an opening on the carrier surface through which particles (e.g., one or more drug molecules) may pass or on which they may be entrapped. According to some aspects, the pores may comprise discrete depressions and/or grooves on the surface of the carrier, and/or may comprise depressions and/or grooves that are connected to provide a porous network. The porous network may comprise, for example, depressions and/or grooves on the surface, and/or the porous network may comprise an inner-network away from the surface of the carrier, for example, an inner-network of channels inside of the carrier. According to some aspects, the carrier pores are configured to trap and/or release one or more drug molecules as described herein.

According to some aspects, the carrier may comprise pores having a macroporous size, for example, a size in the range of 1 to 1000 nm, optionally in the range of 10 to 500 nm, optionally in the range of 10 to 200 nm, and optionally in the range of 20 to 100 nm. Additionally or alternatively, the carrier may comprise pores having a microporous size, for example, a size in the range of 1 to 100 nm, optionally in the range of 1 to 50 nm, optionally in the range of 1 to 20 nm, and optionally in the range of 0.3 to 2 nm. According to some aspects, the pores may have a variable size, that is, the pores may have a size that changes in response to one or more stimuli, such as one or more environmental stimuli. For example, in the case wherein the pore size corresponds to a crosslink density of a hydrogel, the pore size may change in response to hydration and/or dehydration of the hydrogel by an environment. As used herein, the term “environment” should be understood as meaning the environment in which the carrier-drug complex is provided.

It should be understood that either or both of these mechanisms may provide the reversible binding between the one or more drug molecules and the carrier. For example, the carrier may comprise one or more pores configured to physically entrap one or more drug molecules therein, and the pore may also comprise one or more ionic moieties capable of reversibly binding the one or more drug molecules via an ionic interaction. Alternatively or additionally, the carrier may comprise one or more ionic moieties on a surface thereof (i.e., not inside of a pore), and/or pores configured to entrap one or more drug molecules without also providing an ionic interaction.

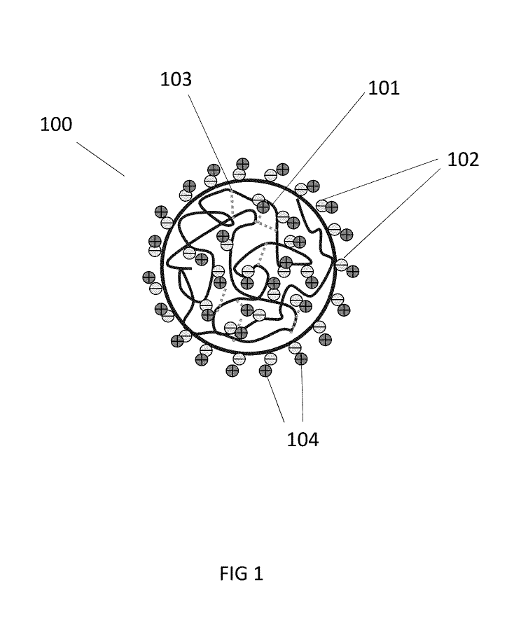

An example of a carrier-drug complex according to the present disclosure is shown in FIG. 1. As shown in FIG. 1, a carrier-drug complex 100 may comprise one or more immobilized polymer chains 101 having negatively charged moieties 102, such as polystyrene sulfonic acid or carbonic acid. The polymer chains 101 may be immobilized by one or more cross-linkers 103, such as divinyl cross-linkers, in order to provide a porous carrier molecule having one or more ionic moieties (i.e., the negatively charged moieties 102). The one or more ionic moieties may be capable of reversibly binding one or more positively charged drug molecules 104 to form the carrier-drug complex 100.

According to some aspects, the carrier may comprise an ionic exchange resin, preferably, a cation-exchange resin. The ionic exchange resin may comprise at least one ionic moiety capable of reversibly binding one or more drug molecules. Examples of ionic exchange resins useful for preparing the carriers of the present disclosure include, but are not limited to, synthetic cationic exchange resins, such as a macroporous polymer matrix with quaternary amine functional group(s) (e.g., Duolite™ AP143-1083 or ScavengePore® phenethyl diethylamine), or a macroporous copolymer matrix with tertiary amine functional group(s) (e.g. Dowex® 66, AMBERLITE™ IRA96, or Amberlyst® A21).

According to some aspects, the carrier may comprise one or more activated carbon molecules. As used herein, the term “activated carbon” refers to carbon processed to have small and/or low-volume pores, for example, as described herein. Examples of activated carbon include, but are not limited to, powdered activated carbon, granular activated carbon, extruded activated carbon, bead activated carbon, impregnated carbon, polymer coated carbon, and woven carbon.

According to some aspects, the carrier may comprise one or more zeolite particles. As used herein, the term “zeolite” refers to aluminosilicate having a three-dimensionally grown skeleton structure generally shown by xM_{2/n}O.Al₂O₃.ySiO₂.zH₂O, written with Al₂O₃ as a basis, wherein M represents an ion-exchangeable metal ion (e.g., the ion of a monovalent or divalent metal); n represents the valence of the metal; x is a coefficient of the metal oxide; y is a coefficient of silica; and z is the number of water of crystallization. Zeolites having different component ratios, fine pore diameters, and specific surface areas are known. According to some aspects, the zeolite particle may be shown by 1 Na₂O.1 Al₂O₃.2.0±0.1 SiO₂.zH₂O.

According to some aspects, the carrier may comprise a gel, such as a hydrogel. As used herein, the term “hydrogel” refers to a gel having a network of crosslinked polymers and a dispersion medium comprising water. It should be understood that the “pores” of a hydrogel may correspond, at least in part, to the polymer crosslink density.

According to some aspects, carrier particle may have a diameter in the range of 500 to 800 μm, optionally in the range of 100 to 500 μm, optionally in the range of 10 to 100 μm, optionally in the range of 1 to 10 μm, and optionally in the range of 0.01 to 1 μm. As used herein, the term “particle” refers to a discrete portion of a composition. For example, a carrier particle may comprise an exchange resin particle, an activated carbon molecule, and/or a zeolite particle. According to some aspects, the carrier may be opaque or otherwise protect at least a portion of the drug from exposure to UV light.

The carrier-drug complex comprises one or more drug molecules which may reversibly bind with the carrier to provide the carrier-drug complex. The drug molecules according to the present disclosure include one or more active portions, that is, one or more portions configured to provide an acceptable effect (e.g., a pharmaceutical and/or antimicrobial effect). Examples of drugs useful according to the present disclosure include, but are not limited to, those containing ionic moieties configured to interact with carrier ionic moieties. According to some aspects, the one or more drug molecules may comprise one or more metal ions, such as silver, copper, zinc, and/or gallium ions, nitric oxide (NO), polymeric cationic antimicrobial molecules such as biguanides (e.g., chlorhexidine salts), including those selected from the group consisting of chlorhexidine gluconate, chlorhexidine acetate, chlorhexidine, chlorhexidine hydrochloride, and biguanides/biguanide derivatives other than chlorhexidine/chlorhexidine salts including alexidine, alexidine salts, polyhexamide, polyhexamide salts, polyaminopropyl biguanide, polyaminopropyl biguanide salts, poly(diallyldimethylammonium chloride) and its polymeric derivatives, and other alkyl biguanides, and combinations thereof. As used herein, the term “derivative” refers to a) a chemical substance that is related structurally to a first chemical substance and derivable from it; b) a compound that is formed from a similar first compound or a compound that can be imagined to arise from another first compound, if one atom of the first compound is replaced with another atom or group of atoms; c) a compound derived or obtained from a parent compound and containing essential elements of the parent compound; or d) a chemical compound that may be produced from an initial compound of similar structure in one or more steps. In particularly preferred embodiments, the drug may comprise any drug useful in pleural effusion therapy. It should be understood that the active portion of the drug molecule may be the same as or different from the ionic moieties configured to interact with carrier ionic moieties. The drug molecules may further comprise one or more additional components as described herein.

The present disclosure also related to methods of making the carrier-drug complexes as described herein. According to some aspects, the carrier-drug complex may be prepared by subjecting one or more carrier molecules to one or more drug molecules such that the one or more drug molecules reversibly bind with the one or more carrier molecules. According to some aspects, the carrier-drug complex may be prepared by providing a first solution containing one or more drug molecules, providing a second solution containing one or more carrier molecules, and combining the two solutions. The combination of the two solutions may provide, for example, wet impregnation of the one or more carrier molecules with the one or more drug molecules.

According to some aspects, upon combination of the carrier molecules and the drug molecules, carrier ionic moieties (e.g., anionic moieties) may release complementary carrier ionic moieties (e.g., cationic moieties) therefrom, and replace the released carrier ionic moieties with the one or more drug molecules (i.e., via an ionic interaction). Additionally or alternatively, one or more drug molecules may become entrapped by carrier pores upon combination of the carrier molecules and the drug molecules.

To prepare the carrier-drug complex, the one or more drug molecules may be provided as part of a precursor compound, wherein one or more portions of the precursor compound is configured to reversibly bind to the carrier molecule to form the carrier-drug complex. For example, the precursor compound may comprise an ionic compound, wherein one or more ions of the ionic compound (e.g., a cation) is configured to reversibly bind with a carrier molecule. Examples of precursor compounds useful according to the present disclosure include, but are not limited to, nitrates (e.g., AgNO₃), silver tetrafluoroborate (e.g., AgBF4), silver hexafluorophosphate (e.g., AgPF₆), silver carbonate (Ag₂CO₃), AgClO₄, [Ag(NH₃)₂]Cl, [Ag(S₂O₃]Cl, [Ag(CN₂)]Cl, CH₃COOAg, salts of organic or polymeric acids (e.g., polyacrylate silver salt), and combinations thereof.

The present disclosure also relates generally to devices having the carrier-drug complexes described herein and methods of making the same. According to some aspects, medical devices according to the present disclosure may be prepared by subjecting a device (e.g., a pleural catheter) to a solution, referred to herein as a “complex solution,” comprising one or more carrier-drug complexes as described herein. According to some aspects, the complex solution may comprise one or more polymers along with the carrier-drug complex(es). Polymers useful according to the present disclosure include, but are not limited to, silicone, polyisobutene, polyisoprene, poly(L-lactide), poly(glycolic acid), polyoxazoline, poly(N-isopropylacrylamide), polyethylene glycol (PEG), cross-linked polyvinyl alcohol, ethyl vinyl acetate, poly 2-hydroxyethyl methacrylate, polypyrrole, dextran, poly (amidoamine), and derivatives and combinations thereof.

The device may be subjected to the complex solution such that the carrier-drug complex(es) and/or the polymer(s) are incorporated as a component of the device, for example, as a coating on one or more surfaces thereof. For example, the medical device may be prepared by dip coating, wherein a device is dipped into the complex solution, or spray coating, wherein the complex solution is sprayed onto a device. Alternatively or additionally, the complex solution may be formulated in an ink having a viscosity suitable for a standard pad printing process, wherein the ink may be printed onto the surface of the device. Alternatively or additionally, the medical device may be prepared using an electrospinning process to load a fiber-like texture containing the carrier-drug complex to form a coating layer.

The medical device according to the present disclosure may be configured such that drug molecules are released therefrom in a stable and/or controllable manner. In particular, the medical device may be configured for insertion into an area of the body (e.g., the pleural cavity) wherein one or more drug molecules may be controllably released into the body.

According to some aspects, release of drug molecules into the body may correspond to ion replacement. For example, ionic components present in the body (e.g., Na+ and/or K+ cations) may replace drug molecules and/or portions thereof (e.g., Ag+ ions) that are reversibly bound to carrier ionic moieties (e.g., anionic moieties). In this way, one or more drug molecules may be released from and/or migrate away from the carrier molecule and/or medical device in a controlled manner. Additionally or alternatively, the drug molecule(s) may comprise one or more components configured to release active portion(s) of the drug molecule(s) in response to one or more stimuli (such as environmental stimuli, including water), for example, by dissolving and/or otherwise degrading (such as biodegrading) such that the active portion(s) of the drug molecule(s) are released therefrom. Examples of dissolvable and/or degradable components include those comprising water soluble polymers such as PEG, poly(2-oxazoline), poly(N-isopropylacrylamide), poly(acrylic acid), polyacrylamide, polyamine, polyethyleneimine, and polyamidoamines; biodegradable polymers such as poly(lactide) and polyglycolide; natural source-extracted degradable polymers such as chitosan, hyaluronic acid and salt form, alginic acid, sodium salt; their derivatives and copolymers; and combinations thereof. According to some aspects, the carrier may alternatively or additionally comprise one or more dissolvable and/or degradable components as described herein such that the carrier is configured to release the drug molecule(s) in response to one or more stimuli. For example, a hydrogel carrier may comprise one or more such components with a crosslinker.

According to some aspects, all or a portion of the drug molecules comprised by the medical device may be released into the body. It should be understood that the release of the drug molecules may correspond to a shift in the equilibrium between drug molecules bound to carrier molecules and drug molecules not bound to carrier molecules.

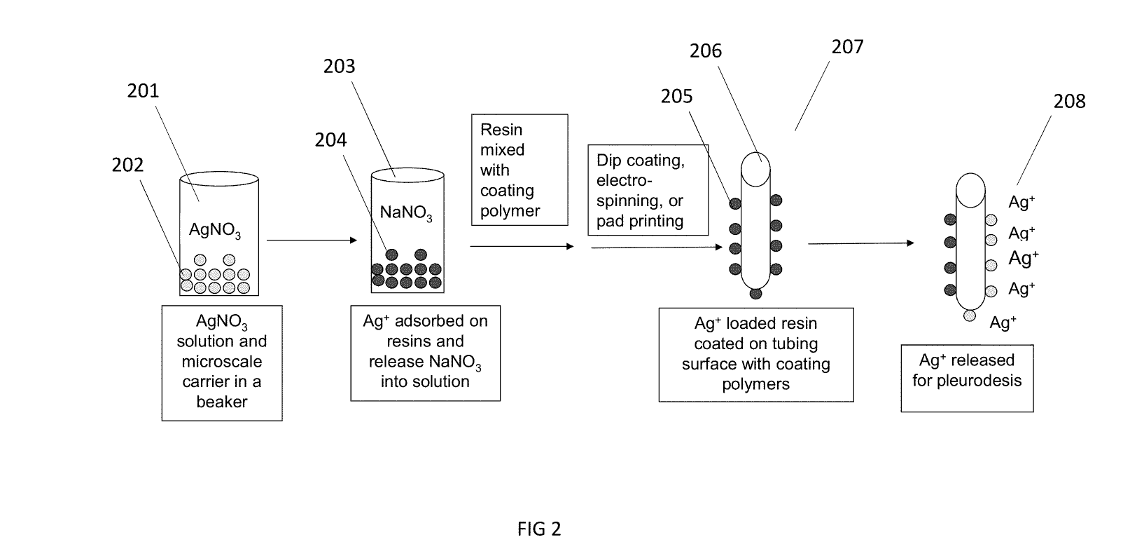

An example medical device and its preparation are shown in FIG. 2. As shown in FIG. 2, a first solution 201 comprising a precursor compound 202, such as AgNO₃, may be combined with a second solution 203 comprising carrier molecules 204 such that drug molecules, e.g., Ag⁺, are adsorbed onto the carrier molecules to provide carrier-drug complexes 205 via wet impregnation. In the example shown in FIG. 2, the wet impregnation results in the formation of NaNO₃ as Ag⁺ molecules replace cationic ionic moieties (i.e., Na⁺ molecules) of the carrier molecules 204.

The carrier-drug complexes 205 optionally may be combined with a polymer in a complex solution, and a device 206 may be coated with the complex solution in order to provide the medical device 207 according to the present disclosure, wherein the carrier-drug complexes 205 are provided on a surface of the device. When the medical device 207 is inserted into the body, one or more drug molecules 208 may be released from the medical device 207.

According to some aspects, the medical device may be configured such that the release of drug molecules is a controlled release. That is, the medical device may be configured such that drug molecules are released therefrom in a selected pattern.

For example, the selected pattern may comprise a constant release rate over a selected period of a time. According to some aspects, the drug molecules may be released from the medical device at a constant rate over a period of 1 hour, optionally 3 hours, optionally 6 hours, optionally 12 hours, optionally 1 day, optionally 2 days, optionally 7 days, optionally 2 weeks, optionally 1 month, optionally 2 months, optionally 6 months, optionally 1 year, or optionally 2 years As used herein, the term “constant rate” refers to a release rate wherein the amount of drug molecules released from the medical device over a first portion of the selected period of time differs from the amount of drug molecules released from the medical device over a second portion of the selected period of time, the second portion being the same length of time as the first portion, by no more than 10 to 90% of the amount of the released drug in the first period of time, optionally no more than 20 to 80%, optionally no more than 30 to 70%, and optionally no more than 40 to 60%. Alternatively or additionally, the medical device may be configured such that drug molecules may be released from the medical device at an increasing or decreasing rate. Alternatively or additionally, the medical device may be configured such that drug molecules may be released from the medical device in one or more bursts (i.e., a period of time wherein the rate of release is different from the constant rate). It should be understood that the medical device may be configured to include any combination of the above release rates. For example, the medical device may be configured such that drug molecules may be released from the medical device at a constant release rate that is interrupted by one or more bursts. According to some aspects, the one or more bursts may occur before, during, or after the period of time when the drug molecules are released at the constant release rate. For example, the burst may occur at a first period of time, wherein the first period of time is followed by a second period of time when drug molecules are released from the medical device at a constant release rate.

According to some aspects, the release of drug molecules from the medical device may be depend, at least in part, on one or more selected characteristics of the medical device. For example, the release may depend, at least in part, on the carrier molecule size, the carrier molecule loading capacity (e.g., the type, size, and/or concentration of pores and/or ionic moieties comprised by the carrier molecule), the concentration of drug molecules loaded onto each carrier molecule, the type of carrier molecule(s), the type of drug molecules(s), and/or the concentration of carrier-drug complexes comprised by the medical device. It should be understood that one or more of these characteristics may be controlled during the production of the medical device to provide a selected release of drug molecules from the medical device. Alternatively or additionally, it should be understood that one or more of these characteristics may depend, at least in part, on one or more environmental stimuli. For example, in the case wherein the pore size of the carrier corresponds to a polymer crosslink density of a hydrogel, the pore size may change in response to hydration and/or dehydration of the hydrogel. In this case, the release of the drug molecule may depend, at least in part, on the capacity of an environment to hydrate the hydrogel. For example, release of the drug molecule may depend, at least in part, on the hydrogel being hydrated such that the pore size is enlarged to a size sufficient to release the drug molecule(s).

According to some aspects, the medical device may be configured to prolong the shelf life of the medical device when compared with similar devices not prepared according to the present disclosure. In particular, the medical device may be configured such that degradation of drug molecules is reduced and/or eliminated when compared with similar devices. For example, the medical device may be configured such that one or more drug molecules is at least partially entrapped within a pore of a carrier molecule, and therefore at least partially protected from UV light as the some of the drugs (e.g., Ag+) are susceptible to photo reduction In this way, the entrapped drug molecule(s) may be at least partially protected from oxidation, thereby at least partially extending product shelf life and/or eliminating the need for UV-shielded device packaging. According to some aspects, product shelf life in non-UV protected packaging at ambient storage conditions may be at least six months, optionally at least one year, optionally at least 1.5 years, optionally at least 2 years, optionally at least 2.5 years, and optionally 3 years.

While the aspects described herein have been described in conjunction with the example aspects outlined above, various alternatives, modifications, variations, improvements, and/or substantial equivalents, whether known or that are or may be presently unforeseen, may become apparent to those having at least ordinary skill in the art. Accordingly, the example aspects, as set forth above, are intended to be illustrative, not limiting. Various changes may be made without departing from the spirit and scope of the disclosure. Therefore, the disclosure is intended to embrace all known or later-developed alternatives, modifications, variations, improvements, and/or substantial equivalents.

Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

Further, the word “example” is used herein to mean “serving as an example, instance, or illustration.” Any aspect described herein as “example” is not necessarily to be construed as preferred or advantageous over other aspects. Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C. Nothing disclosed herein is intended to be dedicated to the public regardless of whether such disclosure is explicitly recited in the claims.

1. A device configured to provide release of a drug, wherein the device comprises a component including a carrier-drug complex, wherein the carrier-drug complex comprises one or more drug molecules reversibly bound to a porous carrier molecule,
wherein the one or more drug molecules is reversibly bound to the porous carrier molecule via entrapment of the one or more drug molecules in a pore of the porous carrier molecule,
wherein the pore has a variable pore size, wherein a size of the variable pore size corresponds to one or more environmental stimuli, and
wherein the porous carrier is a hydrogel, the variable pore size corresponds to a polymer crosslink density of the hydrogel, and the one or more environmental stimuli corresponds to an environment's hydration capacity.

2. The device according to claim 1, wherein the device is configured for use in pleural effusion therapy.

3. The device according to claim 2, wherein the device is a catheter.

4. The device according to claim 1, wherein the component comprises a coating provided on a surface of the device.

5. The device according to claim 4, wherein the coating further comprises one or more polymers.

6. The device according to claim 1, wherein the drug molecule comprises a metal ion.

7. The device according to claim 6, wherein the metal ion comprises a silver ion.

8. A device configured to provide release of a drug, wherein the device comprises a component including a carrier-drug complex, wherein the carrier-drug complex comprises one or more drug molecules reversibly bound to a porous carrier molecule, and wherein the porous carrier molecule comprises one or more carrier ionic moieties configured to:
bind with one or more ionic components present in a human body, or
degrade in the human body.

9. The device according to claim 8, wherein the one or more drug molecules is reversibly bound to the porous carrier molecule via an ionic interaction between the one or more drug molecules and the one or more carrier ionic moieties.

10. The device according to claim 9, wherein the one or more drug molecules is further reversibly bound to the porous carrier molecule via entrapment of the one or more drug molecules in a pore of the porous carrier molecule.

11. The device of claim 10, wherein the pore has a variable pore size, wherein a size of the variable pore size corresponds to one or more environmental stimuli.

12. The device according to claim 11, wherein the porous carrier is a hydrogel and the variable pore size corresponds to a polymer crosslink density of the hydrogel, and wherein the one or more environmental stimuli corresponds to an environment's hydration capacity.

13. The device according to claim 8, wherein the porous carrier is selected from the group consisting of an ionic exchange resin, activated carbon, zeolite, and combinations thereof.

14. A method for making a carrier-drug complex comprising:
providing a first solution containing a precursor compound, wherein the precursor compound comprises one or more drug molecules;
providing a second solution containing one or more porous carrier molecules; and
combining the first and second solutions,
wherein combining the first and second solutions provides wet impregnation of the one or more porous carrier molecules with the one or more drug molecules such that the one or more drug molecules becomes reversibly bound to the one or more porous carrier molecules,
wherein the porous carrier molecule comprises one or more carrier ionic moieties configured to:
bind with one or more ionic components present in a human body, or
degrade in the human body.

15. (canceled)

16. The method according to claim 14, wherein the one or more drug molecules become reversibly bound to the one or more porous carrier molecules via an ionic interaction between the one or more drug molecules and the one or more carrier ionic moieties.

17. The method according to claim 14, wherein the one or more drug molecules become further reversibly bound to the one or more porous carrier molecules via entrapment of the one or more drug molecules in a pore of the one or more porous carrier molecules.

18. A method for making a device comprising:
providing a complex solution comprising a carrier-drug complex, wherein the carrier-drug complex had been prepared according to the method of claim 14;
providing a device;
subjecting the device to the complex solution to provide a coating on a surface of the device, wherein the coating comprises the carrier-drug complex.

19. The method of claim 18, wherein subjecting the device to the complex solution comprises dip coating, pad printing, electrospinning, or a combination thereof.

20. The method of claim 18, wherein the complex solution further comprises a polymer.

21. The method of claim 18, wherein the device is configured for use in pleural effusion therapy.

22. The method of claim 18, wherein the drug molecule comprises a metal ion.
